(19) United States
(12) Patent Application Publication
(10) Pub. No.: US 2026/0237491 A1
(43) Pub. Date: Aug. 13, 2026

(54) METHOD AND SYSTEM FOR SURVEILLANCE OF HEALTHCARE-ASSOCIATED SYSTEMS IMPLEMENTING ARTIFICIAL INTELLIGENCE

(71) Applicant: RAD AI, Inc., San Francisco, CA (US)

(72) Inventors: Doktor Gurson, Honolulu, HI (US); Jeffrey Chang, Prosper, TX (US)

(73) Assignee: RAD AI, Inc., San Francisco, CA (US)

(21) Appl. No.: 19/532,180

(22) Filed: Feb. 6, 2026

Related U.S. Application Data
(60) Provisional application No. 63/756,751, filed on Feb. 10, 2025.

(51) Int. Cl. G16H 30/40; G16H 30/20; G16H 40/20; G16H 50/20

(52) U.S. Cl. CPC G16H 30/40; G16H 30/20; G16H 40/20; G16H 50/20

(57) ABSTRACT

A method for production monitoring and/or surveillance includes: retrieving a set of outputs of a healthcare system including artificial intelligence (AI) architecture for generating the set of outputs; generating an analysis associated with the set of outputs; and executing an action in response to a feature of the analysis, thereby providing surveillance of the healthcare system. The inventions described function to provide architecture and processes for safely distributing and evaluating AI tools in the context of healthcare systems and other systems. Provision of high quality and reliable surveillance, according to the inventions described, can be used for auditing and/or clearance of such AI tools in accordance with regulation standards.

### CROSS-REFERENCE TO RELATED APPLICATIONS

This application claims the benefit of U.S. Provisional Application No. 63/756,751 filed 10 Feb. 2025, which is incorporated in its entirety by this reference.

### TECHNICAL FIELD

This invention relates generally to the healthcare field, and more specifically to a new and useful system and method for AI healthcare system surveillance and monitoring in the healthcare field.

### BRIEF DESCRIPTION OF THE FIGURES

FIGS. 1A and 1B are a schematic representations of an embodiment of a method and associated system for monitoring of AI-supported healthcare systems.

FIGS. 2A-2G depict embodiments of healthcare system and method features that can be monitored using the invention(s) described.

FIG. 3 depicts a schematic representation of an embodiment of a system for monitoring of AI-supported healthcare systems.

### DETAILED DESCRIPTION

The following description of the embodiments of the invention is not intended to limit the invention to these embodiments, but rather to enable any person skilled in the art to make and use this invention.

### 1. Overview

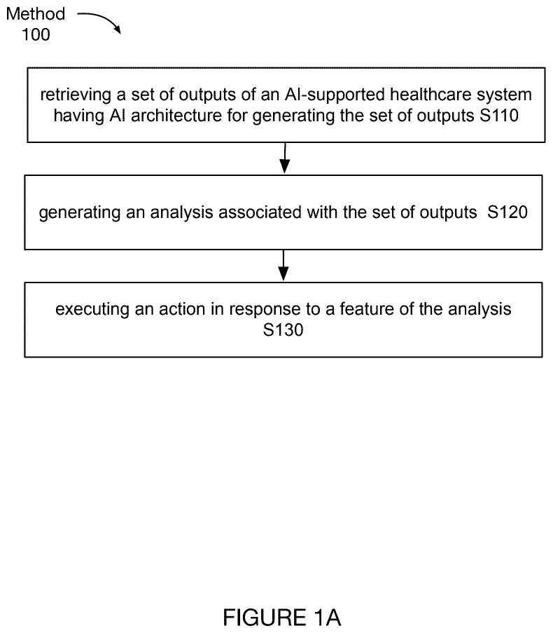

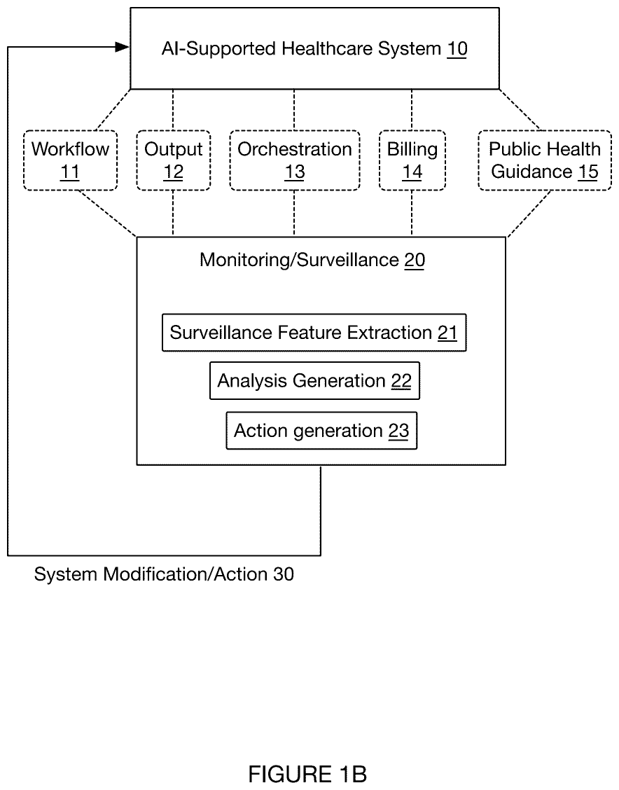

As shown in FIGS. 1A and 1B, an embodiment of a method 100 can include: retrieving a set of outputs of a healthcare system including artificial intelligence (AI) architecture for generating the set of outputs S110; generating an analysis from the set of outputs S120; and executing an action in response to a feature of the analysis S130, thereby providing surveillance of the healthcare system. In examples, the method 100, described in more detail in Section 3 below, can function to provide surveillance of deployed AI radiology systems, in relation to diagnostics, triage, and follow-up actions. In examples, the method 100 can be applied to one or more of: continuous monitoring workflows; orchestration of image data; orchestration of test data; orchestration of analytics; orchestration of quality metrics data; orchestration of associated workflows for both health systems and physician practice groups; orchestration of associated workflows for entities in healthcare-associated operations, including payers, life sciences companies, pharmaceutical companies, therapeutics companies, etc.; and other workflows.

FIG. 1B depicts a schematic including an AI-supported healthcare system 10; variations of processes and/or returned components (e.g., workflow 11, output 12, orchestration process 13, billing-associated output 14, public health guidance output 15, etc.) generated in association with the AI-supported healthcare system 10; monitoring and surveillance architecture 20 including functionality for extracting surveillance features 21 (described in more detail below), generating analyses 22, and generating actions 23; and system modification iterative actions 30 that can be used to maintain or otherwise improve performance of the AI-supported healthcare system 10.

Variations of the method 100 can involve surveillance and production monitoring, with fully automated, semi-automated, and/or manually-driven production monitoring of AI models and solutions in the healthcare space, with specific examples involving AI radiology systems, as described below. The method 100 can be implemented by embodiments, variations, and examples of system components described in Section 4 below.

### 2. Technical Advantages and Examples

The systems and methods described can provide architecture and frameworks for safely distributing AI tools in the context of healthcare systems (e.g., radiology platforms, etc.). Provision of high quality and reliable surveillance, according to the methods and systems described, can be used for auditing and/or clearance of such AI tools in accordance with regulation standards.

In variations, healthcare systems monitored using the invention(s) described can include radiology platforms with architecture for automatically generating an impression section of a radiology report, radiology platforms with architecture for automatically annotating radiology image data of a picture archiving and communication system (PACS), other systems described, and/or other smart-PACS functionality; radiology platforms with architecture for providing an automatic follow-up service for a patient in response to an actionable finding for the patient, and/or other radiology platforms. Embodiments, variations, and examples of radiology platform functionality are described in U.S. application Ser. No. 19/249,015 filed on 25 Jun. 2025, U.S. application Ser. No. 18/757,020 filed on 27 Jun. 2024, U.S. application Ser. No. 18/108,615 filed on 11 Feb. 2023, U.S. application Ser. No. 19/174,282 filed on 9 Apr. 2025, U.S. application Ser. No. 19/235,195 filed on 11 Jun. 2025, U.S. application Ser. No. 19/235,790 filed on 11 Sep. 2025, and U.S. application Ser. No. 18/952,147 filed on 19 Nov. 2024, which are each herein incorporated in its entirety by this reference.

In additional variations, healthcare systems monitored using the invention(s) described can include clinical decision support and diagnostic reasoning systems, which analyze patient data (e.g., including symptoms, labs, vitals, imaging summaries, genomics, history, etc.) to assist clinicians with diagnosis, differential ranking, and care recommendations. Such systems can be standalone or embedded in electronic health record (HER) workflows.

In additional variations, healthcare systems monitored using the invention(s) described can include pathology and laboratory medicine systems, which include architecture for processing tissue samples/slides, cytology images, and lab result patterns to identify malignancies, grade tumors, detect infections, and predict disease progression. Such systems can assist pathologists by highlighting regions of interest, quantifying features humans eyeball imperfectly, and reducing inter-observer variability.

In additional variations, healthcare systems monitored using the invention(s) described can include predictive analytics and early warning platforms that are structured to continuously monitor patient data streams to predict adverse events before they happen. Such systems implement AI architecture to identify trends humans are not adapted to reliably monitor.

In additional variations, healthcare systems monitored using the invention(s) described can include personalized and precision medicine platforms that are structured to integrate data from multiple sources (e.g., genomics, transcriptomics, proteomics, imaging, and clinical history) to tailor treatments to individual patients.

In additional variations, healthcare systems monitored using the invention(s) described can include clinical workflow automation and documentation intelligence systems, remote monitoring and digital therapeutics systems, population health and health system optimization systems, non-radiology imaging support systems (e.g., in the context of ophthalmology, in the context of dermatology, etc.), surgical guidance systems (e.g., presurgical planning systems with AI-assisted computer vision architecture), medical training systems (surgical and non-surgical), AI-supported biometric monitoring device platforms, AI-supported mental healthcare providing platforms, AI-supported therapeutic platforms, AI-supported drug discovery platforms, AI-supported diagnostic devices, AI-supported life sciences measurement tool platforms, and/or other healthcare or healthcare-adjacent systems.

Healthcare systems monitored using the invention(s) described can be configured for human subjects and/or non-human subjects. In one example, healthcare systems monitored using the methods and systems described, where the healthcare systems are associated with non-human subjects, can include veterinary care systems. In one example, healthcare systems monitored using the methods and systems described, where the healthcare systems are associated with non-human subjects, can include environmental health-affiliated systems (e.g., associated with air quality, associated with water quality, associated with contaminants, associated with building regulations, associated with environmental impact analyses, etc.).

Applications of the invention(s) described can further extend beyond healthcare, with respect to providing surveillance and modifying system architecture and behavior in order to improve performance of such systems.

The systems and methods described can provide one or more of: continuous monitoring workflows for evaluating performance of AI-supported healthcare systems; orchestration of image data, test data, analytics data, and/or other quality metrics data and associated workflows for both health systems and practice groups, as well as other entities (e.g., payers, life sciences companies, pharmaceutical industry entities, etc.); full automation production monitoring of AI models and solutions; partial automation production monitoring of AI models and solutions partly automated; and manual-driven driven production monitoring of AI models and solutions.

In examples, the methods and systems described can return population-level analyses in relation to monitoring and surveillance applications of the inventions described. In one example, outputs returned by the invention(s) can include population-level and sub-population-level characterizations of performance of healthcare entities (e.g., radiologists, clinicians, facility personnel, other entities associated with providing healthcare, etc.) and/or entities (e.g., patients, etc.) receiving care from such healthcare entities. Characterizations can provided at various levels of abstraction, involve statistical measures at different resolutions and/or across various time points. In examples, variations from baseline (e.g., variations reported from a site, subpopulation, specific demographic, etc.) can be flagged and/or reported as excursion from baseline.

In examples, the methods and systems described can collect and generate statistics for healthcare entities (e.g., radiologists, clinicians, facility personnel, other entities associated with providing healthcare, etc.) in relation to monitoring and surveillance applications of the inventions described. In such examples, the inventions can detect entities or entity groups (e.g., individuals or groups of radiologists or clinicians) who are or may be over-riding use of the AI-supported platform. Such analyses can determine if such behaviors were concentrated in a particular individual/site/population, and then to target interventions to such detected particular individuals/sites/populations in order to prevent further adverse behavior and/or to improve performance by the identified particular individuals/sites/populations, by executing actions described in further detail below.

In examples, the methods and systems described can process returned diagnostics that prompted additional follow up studies (e.g., in the context of radiology findings and reports, in the context of other clinical diagnostics, etc.) that provide more definition to aspects of the diagnostics and report those results. Exemplary follow up studies can include additional pathology screenings generated through sampling (e.g., biopsies, blood tests, other tests involving biological sampling, etc.). Such interventions can thus provide continuity of support for patients and/or other subjects, as described in Applications incorporated by reference above.

In examples, the methods and systems described can process auxiliary data (e.g., data associated with hospitalizations, data associated with patient mortality, data associated with patient prognoses, data associated with other patient outcomes), and generate comparative outputs including relevant metrics (e.g., metrics comparing results from auxiliary data and results from the AI-supported platform). Comparative analyses can be segmented according to any grouping (e.g., population level of abstraction, subpopulation level of abstraction, demographic grouping, etc.). Methods described can thus further include processing auxiliary comparative data as training data to refine model architecture structured to provide surveillance of the healthcare systems described.

In examples, the methods and systems described can further return economic impact reports, with respect to projections or other estimates of the economic impact of deployments (e.g., in relation to analyses of which follow up studies were prompted as well as associated costs of follow up studies or treatments, in relation to the longer-term costs associated with care for diagnosed and/or stratified patients with a particular diagnostic, etc.). Economic cost estimates can be based upon cost tables for various treatments (e.g., provided by Centers for Medicare and Medicaid services (CMS), provided by other cost schedules, etc.), and methods described can process historical economic cost data as training data to refine model architecture for projecting future cost implications of applying or not applying modifications to the monitored healthcare systems described, in relation to executed actions described below.

In examples, the methods and systems described can incorporate generative AI architecture for generation of reports and/or other outputs for the Food and Drug Administration (FDA) and payers. The methods and systems can further transform reports and other outputs into summaries (e.g., text summaries), tabular reports with auto generated data analysis code (e.g., in R, in Python, etc.) for further processing (e.g., by a regulator, by a payer, etc.). Such reports can thus summarize performance of monitored healthcare systems and/or indicate outputs of the methods and systems described in a useful format.

In relation to practical applications, the methods and systems described can provide significant reductions in economic impact, in relation to radiologist time and to organizations involving radiologists and/or other care-providing entities, in terms of revenue capture. The methods and systems can also provide significant reductions in economic impact at the payer-level, as a result of automating monitoring and surveillance of AI-supported care-providing platforms.

In examples, significant reductions in economic impact can be attributed to dictation reduction performance, with respect to radiologist or other healthcare-providing entity time involved to perform tasks. Dictation reduction performance, in terms of dictation reduction percentage, for instance, can include: dictation of 20% fewer words by clinicians/radiologists (e.g., in order to generate a report from a clinical session), dictation of 22% fewer words by clinicians/radiologists (e.g., in order to generate a report from a clinical session), dictation of 24% fewer words by clinicians/radiologists (e.g., in order to generate a report from a clinical session), dictation of 26% fewer words by clinicians/radiologists (e.g., in order to generate a report from a clinical session), dictation of 28% fewer words by clinicians/radiologists (e.g., in order to generate a report from a clinical session), dictation of 30% fewer words by clinicians/radiologists (e.g., in order to generate a report from a clinical session), dictation of 32% fewer words by clinicians/radiologists (e.g., in order to generate a report from a clinical session), dictation of 34% fewer words by clinicians/radiologists (e.g., in order to generate a report from a clinical session), dictation of 36% fewer words by clinicians/radiologists (e.g., in order to generate a report from a clinical session), dictation of 38% fewer words by clinicians/radiologists (e.g., in order to generate a report from a clinical session), dictation of 40% fewer words by clinicians/radiologists (e.g., in order to generate a report from a clinical session), dictation of 42% fewer words by clinicians/radiologists (e.g., in order to generate a report from a clinical session), dictation of 44% fewer words by clinicians/radiologists (e.g., in order to generate a report from a clinical session), dictation of 46% fewer words by clinicians/radiologists (e.g., in order to generate a report from a clinical session), dictation of 48% fewer words by clinicians/radiologists (e.g., in order to generate a report from a clinical session), dictation of 50% fewer words by clinicians/radiologists (e.g., in order to generate a report from a clinical session), or a lower percentage of words dictated by clinicians/radiologists (e.g., in order to generate a report from a clinical session).

In examples, significant reductions in economic impact can be attributed to speed performance, with respect to radiologist or other healthcare-providing entity time involved to perform tasks. Increased speed performance can include: 30 minutes saved (e.g., per clinical shift, per caretaking shift, per radiologist shift), thus providing ~6% improved speed in relation to standard systems; 35 minutes saved (e.g., per clinical shift, per caretaking shift, per radiologist shift), thus providing ~7% improved speed in relation to standard systems; 40 minutes saved (e.g., per clinical shift, per caretaking shift, per radiologist shift), thus providing ~8% improved speed in relation to standard systems; 45 minutes saved (e.g., per clinical shift, per caretaking shift, per radiologist shift), thus providing ~9% improved speed in relation to standard systems; 50 minutes saved (e.g., per clinical shift, per caretaking shift, per radiologist shift), thus providing ~10% improved speed in relation to standard systems; 55 minutes saved (e.g., per clinical shift, per caretaking shift, per radiologist shift), thus providing ~11% improved speed in relation to standard systems; 60 minutes saved (e.g., per clinical shift, per caretaking shift, per radiologist shift), thus providing ~12% improved speed in relation to standard systems; 65 minutes saved (e.g., per clinical shift, per caretaking shift, per radiologist shift), thus providing ~13% improved speed in relation to standard systems; 70 minutes saved (e.g., per clinical shift, per caretaking shift, per radiologist shift), thus providing ~14% improved speed in relation to standard systems; 75 minutes saved (e.g., per clinical shift, per caretaking shift, per radiologist shift), thus providing ~15% improved speed in relation to standard systems; 80 minutes saved (e.g., per clinical shift, per caretaking shift, per radiologist shift), thus providing ~16% improved speed in relation to standard systems; or greater speed performance.

In examples, significant reductions in economic impact can be attributed to detection sensitivity, with respect to detection of various clinical indications, thereby reducing resource waste. In examples, the systems and methods described can produce: greater than 70% sensitivity (e.g., in detection of a clinical indication), greater than 71% sensitivity (e.g., in detection of a clinical indication), greater than 72% sensitivity (e.g., in detection of a clinical indication), greater than 73% sensitivity (e.g., in detection of a clinical indication), greater than 74% sensitivity (e.g., in detection of a clinical indication), greater than 75% sensitivity (e.g., in detection of a clinical indication), greater than 76% sensitivity (e.g., in detection of a clinical indication), greater than 77% sensitivity (e.g., in detection of a clinical indication), greater than 78% sensitivity (e.g., in detection of a clinical indication), greater than 79% sensitivity (e.g., in detection of a clinical indication), greater than 80% sensitivity (e.g., in detection of a clinical indication), greater than 85% sensitivity (e.g., in detection of a clinical indication), greater than 90% sensitivity (e.g., in detection of a clinical indication), or greater sensitivity.

In examples, significant reductions in economic impact can be attributed to clinical indication detection specificity, with respect to detection of various clinical indications, thereby reducing resource waste. In examples, the systems and methods provided: greater than 80% specificity (e.g., in detection of a clinical indication), greater than 82% specificity (e.g., in detection of a clinical indication), greater than 84% specificity (e.g., in detection of a clinical indication), greater than 86% specificity (e.g., in detection of a clinical indication), greater than 88% specificity (e.g., in detection of a clinical indication), greater than 90% specificity (e.g., in detection of a clinical indication), greater than 92% specificity (e.g., in detection of a clinical indication), greater than 94% specificity (e.g., in detection of a clinical indication), greater than 96% specificity (e.g., in detection of a clinical indication), greater than 98% specificity (e.g., in detection of a clinical indication), or greater specificity.

In relation to performance achievements attributed to improved monitoring and surveillance, the methods and systems described can provide a significant reductions in resource use (e.g., financial costs) associated with monitoring of AI-supported healthcare-providing platforms, which benefits facilities, patients, and payers. The systems and methods can thus provide a streamlined solution that reduces healthcare system expenditures associated with one or more of: AI-platform monitoring costs, costs associated with errors in diagnosis and treatment, costs associated with medical billing errors, or other associated costs.

Further advantages can be provided by the system and method disclosed herein.

### 3. Method

As shown in FIG. 1A, an embodiment of a method 100 includes: retrieving a set of outputs of a healthcare system including artificial intelligence (AI) architecture for generating the set of outputs S110; generating an analysis associated with the set of outputs S120; and executing an action in response to a feature of the analysis S130, thereby providing surveillance of the healthcare system. The method 100 functions to provide architecture and processes for safely distributing AI tools in the context of healthcare systems (e.g., radiology platforms, etc.). Provision of high quality and reliable surveillance, according to the methods described, can be used for auditing and/or clearance of such AI tools in accordance with regulation standards.

Embodiments, variations, and examples of the method 100 can be executed using components of systems described in Section 4 below and/or components as described in any or all of: U.S. application Ser. No. 16/688,623, filed 19 Nov. 2019; U.S. application Ser. No. 17/020,593, filed 14 Sep. 2020; U.S. application Ser. No. 17/690,751, filed 9 Mar. 2022; U.S. application Ser. No. 18/215,354, filed 28 Jun. 2023; U.S. application Ser. No. 17/649,213, filed 28 Jan. 2022; U.S. application Ser. No. 18/374,535, filed 28 Sep. 2023; U.S. application Ser. No. 18/374,526, filed 28 Sep. 2023; U.S. application Ser. No. 18/952,233, filed 19 Nov. 2024; and U.S. application Ser. No. 18/952,147, filed 19 Nov. 2024, each of which is incorporated in its entirety by this reference.

### 3.1 Method—Retrieving Outputs for Monitoring and Surveillance

Step S110 recites: retrieving a set of outputs of a healthcare system, where the healthcare system includes artificial intelligence (AI) architecture for generating the set of outputs. Step S110 functions to export or otherwise retrieve data from which surveillance and monitoring-associated characterizations can be generated in subsequent steps of the method 100. Retrieval can include providing one or more interfaces with one or more of: a set of databases (e.g., electronic health record (EHR) database, electronic medical record (EMR) databases, radiology information system (RIS) databases, clinical information system (CIS) databases, picture archiving and communication system (PACS) databases, etc.).

Additionally or alternatively, retrieving outputs can include providing one or more interfaces with elements described in Section 4 below, including one or more of: a reporting platform; alternative image viewing and image storage platforms; a speech recognition platform; a radiology worklist; a Health Information System (HIS) platform; a Laboratory Information System (LIS) platform; vendor-neutral archive (VNA) components; ontologies (e.g., radiological or other clinical ontology database); and/or any other database, storage, server, and/or software tools.

Step S110 can be performed using a computing system comprising an interface with the healthcare system, where the interface comprises structures for transmitting data/digital objects, in a secure manner, between the computing system and other participating system. In variations, the interface can facilitate performance of Step s110 by providing a secure data-transmission interface configured to enable controlled and protected exchange of information between distinct systems, such that Step S110 is performed directly between systems, without involvement or organization of human behavior. In variations, communications through the interface can be implemented using application programming interfaces (APIs) that define standardized communication protocols, data formats, and access methods while abstracting underlying system architecture. As a secure interface, the interface of Step S110 can incorporate authentication and authorization mechanisms, including credential-based access controls and role-based permissions, in combination with encrypted communication channels to protect data confidentiality and integrity during transmission. Additional safeguards, such as input validation, transaction logging, and request throttling, may be employed to mitigate unauthorized access, data corruption, and misuse, with respect to communications involving the interface. Collectively, these features allow the interface to function as a secure, well-defined boundary for interoperable system integration while maintaining compliance with applicable security and data protection requirements.

Retrieval can include retrieval of data of any suitable data structure or format.

In embodiments, the healthcare system architecture can be a platform for radiologists, where the platform includes AI tools for optimizing radiology workflows and automating all or portions of tasks performed by radiologists. Embodiments, variations, and examples of platform aspects are described in Applications incorporated by reference above. Alternatively, the healthcare system including AI architecture can include other healthcare-providing or otherwise healthcare-associated platforms structured to facilitate delivery of care to patients or other entities receiving therapy (e.g., in a regulated format, in a non-regulated format), where embodiments, variations, and examples of other healthcare providing or healthcare-adjacent systems are described above.

In embodiments, AI architecture of the healthcare system can include language models configured for natural language processing and/or to produce language (e.g., words, text, strings of text, sentences, etc.) (e.g., as part of a natural language processing [NLP] task) that can be used in and/or form a radiology report. In a specific example, models can include: one or more transformers and/or transformer systems (e.g., Bidirectional Encoder Representations from Transformers [BERT], Generative Pre-Trained Transformer [GPT], etc.); a transformer with any suitable number and/or arrangement of encoders and decoders (e.g., arranged in a sequential and/or parallel arrangement); and/or any other suitable transformers or models. In a second specific example, models can include: one or more non-transformer based models (e.g., deep learning-based models such as Mamba, sequence modeling techniques, state space models, etc.); and/or any other large language models and/or other suitable models. In preferred variants, the language model includes a large language model (LLM), wherein the LLM includes one or more transformers. As such, the LLM model architecture can include deep neural networks or other forms trained on large-scale, heterogeneous datasets using transformer-based architectures to perform tasks such as text generation, semantic understanding, summarization, and decision support through probabilistic sequence modeling.

Additionally or alternatively, the language model can include multiple models (e.g., multiple LLMs, multiple LLMs applied in series, etc.) and/or any other models. Additionally or alternatively, the language model(s) implemented can include small language models that are trained on a subset of scenario-specific training data. Additionally or alternatively, the set of models can include any other models.

Alternatives to LLMs implemented by the healthcare systems described can include smaller task-specific neural networks, classical machine learning models (e.g., support vector machines, gradient-boosted trees), rule-based expert systems, symbolic reasoning engines, and hybrid neuro-symbolic frameworks that combine statistical learning with deterministic logic. Additional architectures can involve retrieval-based systems, knowledge graphs, or Bayesian inference models that prioritize explainability, computational efficiency, or deterministic behavior over generative flexibility. Selection among LLMs and alternative model architectures may be based on factors including accuracy requirements, interpretability, latency, computational cost, data governance constraints, and suitability for deployment in regulated or resource-constrained environments.

In variations, AI architecture of the healthcare system can include a multimodal model. In variations and examples, the multimodal model can include a large language model (LLM) and a vision encoder structured as a language-aligned image encoder integrated, by way of an adapter, onto a fixed LLM. The multimodal model can be structured to receive a diverse set of inputs (e.g., dictation data, text data, images, audio, etc.), and is trained and re-trained across diverse input types simultaneously, in order to facilitate automation of all or a portion of tasks associated with providing healthcare. The multimodal models described also integrate advanced vision models with LLM architecture, which involve transformer-based artificial intelligence (AI) models that are trained and re-trained on large datasets and can generalize to a range of clinical tasks (e.g., zero-shot image-to-text generation upon receipt of natural language instruction inputs). Training of such models addresses limitations of vision-only models and are trained on multimodal data (e.g., clinical images with paired free-text reports that meet various accuracy and billing standards). Multimodal model structures described are capable of generating solutions associated with the long-tails of diagnoses in the clinical domain and/or outside of the clinical domain. In particular, long-tails of diagnoses describe situations where a small number of conditions are routinely observed, but the majority are rare.

In the specific example, an LLM can include a version of the Pathways Language Model (e.g., PaLM, PaLM2, etc.). Variations of the LLM can include a version of a Language Model for Dialogue Applications (LaMDA), a Gemini model (e.g., a decoder-only transformer), a GPT model, a Llama model, a GLM model, a Claude model, a Reka Flash model, a Qwen model, a Grok model, a Molmo model, a Jamba model, a DeepSeek Coder model, an Athene model, a Phi-3 model, a Command-R-Plus model, an InternLM model, a Yi-Large model, a Mixtral of Experts model, a Gemma model, a Nemotron model, and/or another suitable model.

The multimodal model can have a context length of: up to 3,000,000 tokens, up to 2,000,000 tokens, up to 1,000,000 tokens, up to 500,000 tokens, up to 100,000 tokens, up to 90,000 tokens, up to 80,000 tokens, up to 70,000 tokens, up to 60,000 tokens, up to 50,000 tokens, up to 40,000 tokens, up to 35,000 tokens, up to 33,000 tokens, up to 30,000 tokens, up to 25,000 tokens, or another suitable number of tokens. Each context window can contain multiple forms of input, and different modes can be interleaved without requirement to be presented in a fixed order, allowing for a multimodal conversation. Input images can be of different resolutions. The multimodal model can have sparse mixture-of-experts architecture.

In specific examples, the multimodal model can be trained and re-trained (e.g., with generated outputs) for automation of healthcare-providing tasks, using image data (e.g., from various types of images described) paired with corresponding free-text clinical reports (e.g., radiology reports), where the clinical reports include reports generated by clinicians (e.g., radiologists) and/or modified by clinicians post-automatic generation of a candidate report.

However, the AI architecture of the healthcare system can be otherwise structured and/or include other forms of architecture for automation of other tasks.

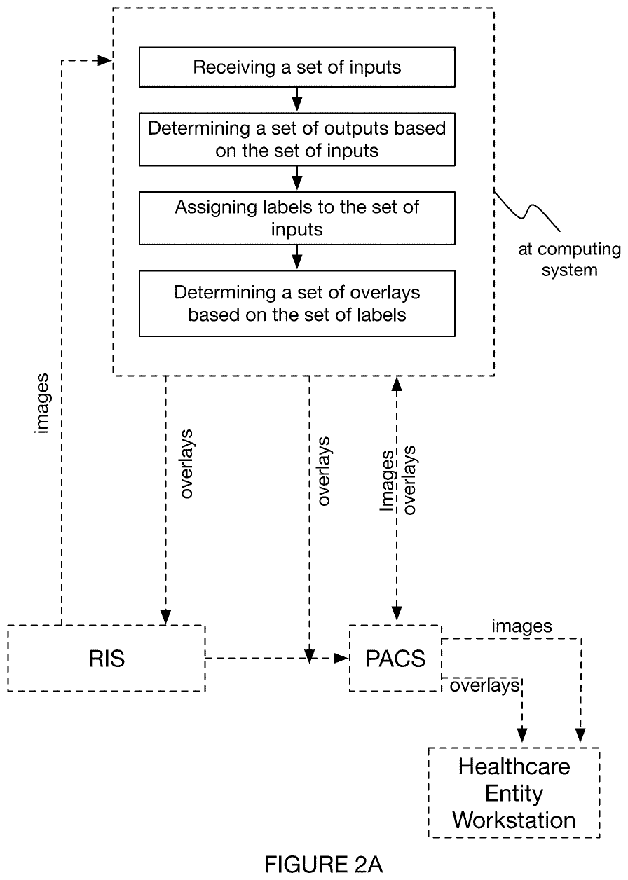

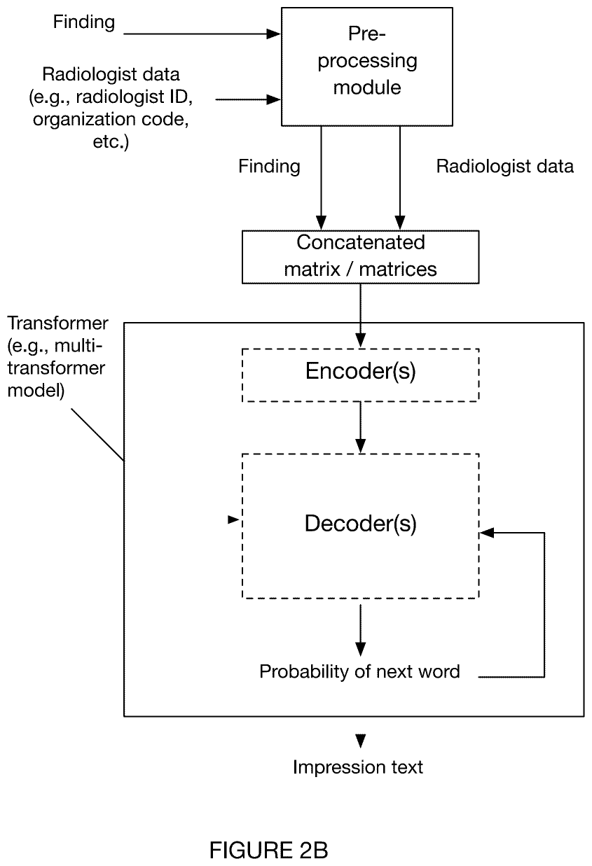

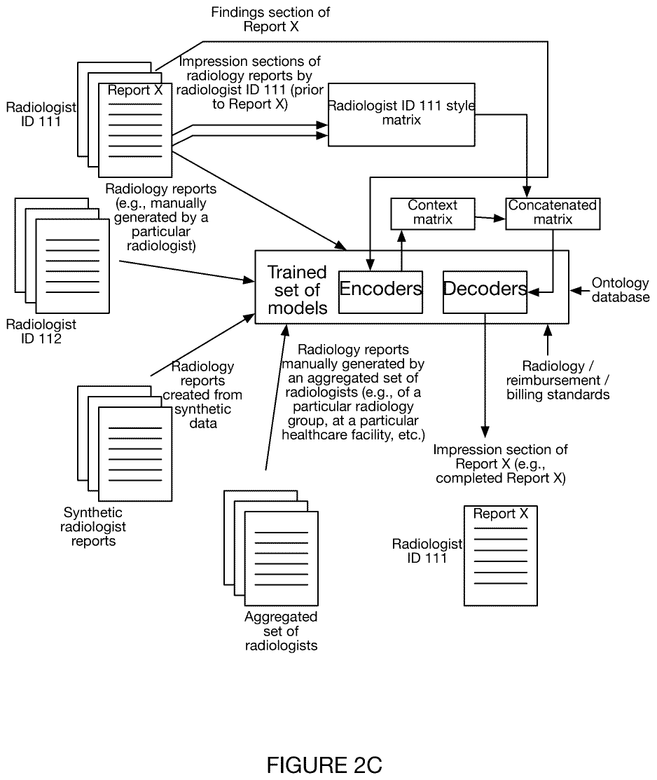

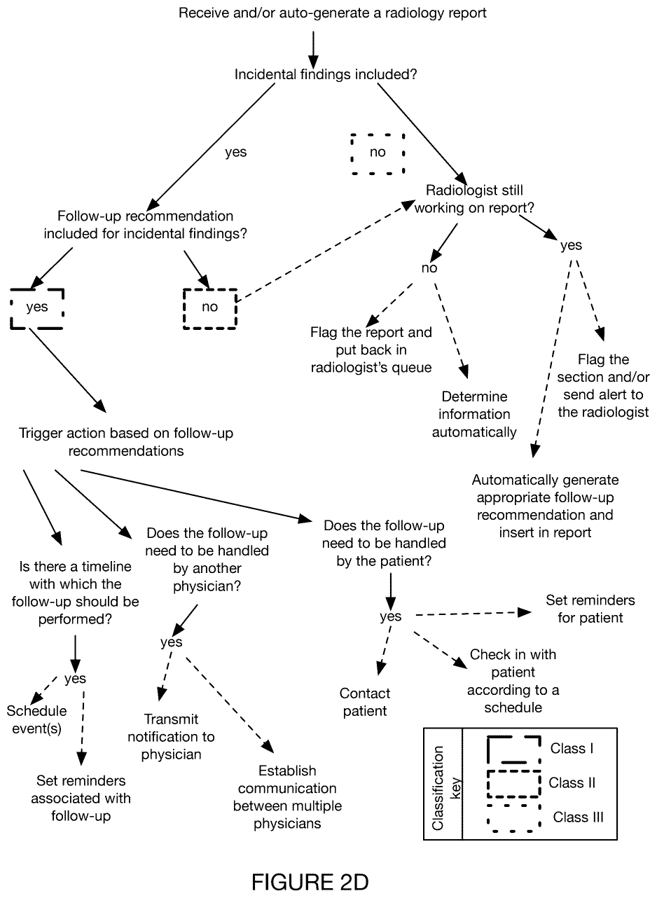

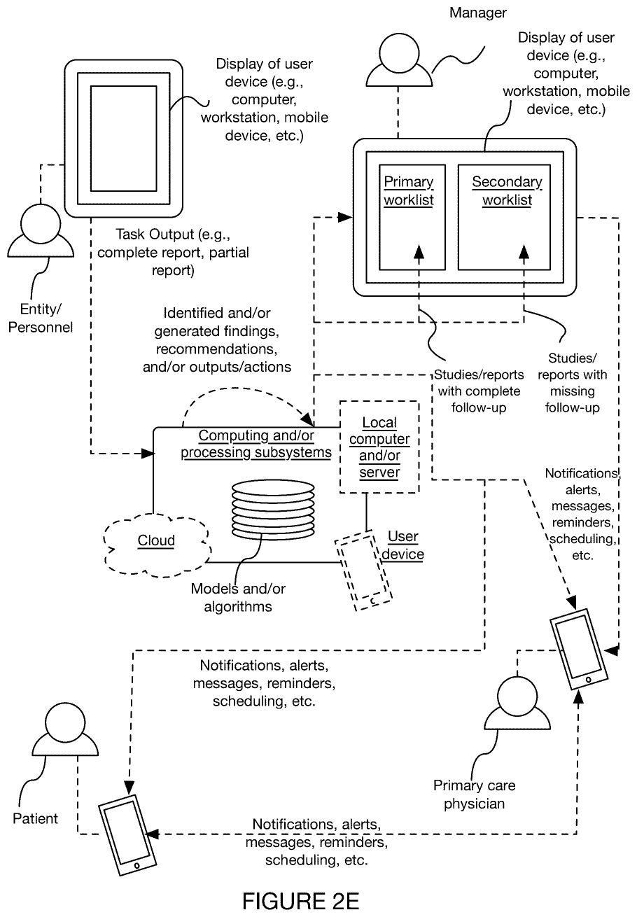

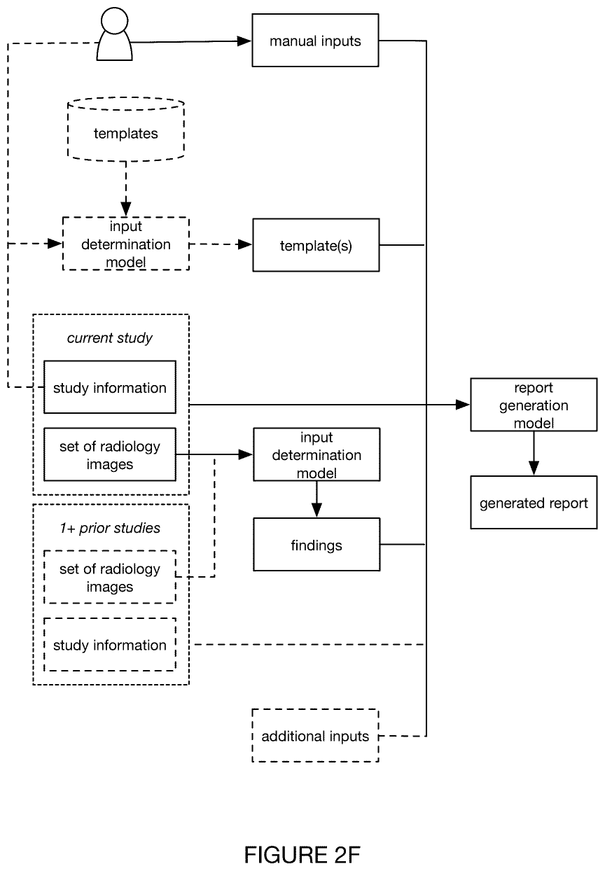

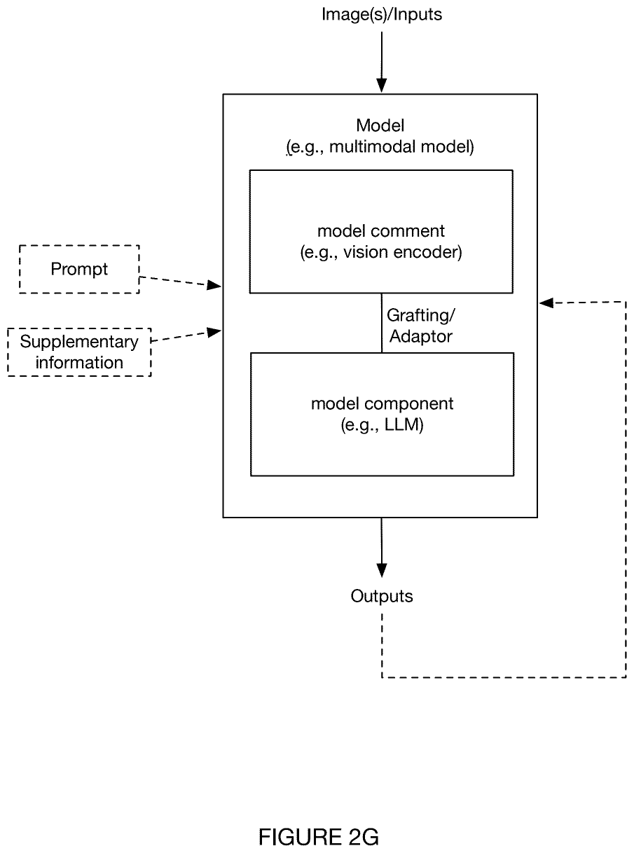

In relation to retrieving outputs in Step S110 using the computing system, the set of outputs can include one or more of: all or portions of radiology reports; all or portions of impressions of radiology reports; all or portions of findings; all or portions of annotated images; all or portions of outputs pertaining to dashboards or other interactions with user interfaces (e.g., dashboards) for radiologist workflows; all or portions of radiology worklist events; all or portions of outputs pertaining to patient follow-up systems; radiologist performance metric values; clinician performance metric values; facility performance metric values; aspects of diagnostics for a facility, radiology group, radiologist, or other associated entity; billing data; billing error data; and/or other suitable outputs. Aspects of outputs are described in Applications incorporated by reference above, and some aspects are depicted in FIGS. 2A-2G.

Outside of the context of radiology, the set of outputs of Step S110 can include one or more of: all or portions of reports (e.g., clinical reports, session reports, treatment reports, follow-up reports); communications between caretaking entities; communications between caretaking entities and individuals receiving care; communications by or to individuals receiving care; data pertaining to aspects of treatment in a physical and/or virtual environment; all or portions of findings; all or portions of annotated images, video, and/or other data associated with characterizations or diagnostics; all or portions of outputs pertaining to dashboards or other interactions with user interfaces (e.g., dashboards) for caretaker workflows; all or portions of caretaker worklist events; all or portions of outputs pertaining to patient follow-up systems; clinician performance metric values; facility performance metric values; aspects of diagnostics for a facility, clinical group, caretaker, or other associated entity; billing data; billing error data; and/or other suitable outputs.

Information can be retrieved in a manner that conforms to privacy regulation standards (e.g., with suitable encryption protocols). Retrieval can thus involve performing authentication processes for accessing or retrieving encrypted data, with or without decryption of personal identifying information.

### 3.2 Method—Generating Analyses of Outputs for Monitoring and Surveillance

Step S120 recites: generating an analysis associated with the set of outputs S120. Step S120 functions to process outputs of Step S110 with various transformation operations, in order to generate one or more analyses that can be used to guide execution of monitoring and surveillance actions in Step S130. Step S120 can be performed at the computing system used for Step S110, or alternatively, another computing system.

Step S120 can provide analyses of orchestration of image data, test data, analytics data, and/or other quality metrics data and associated workflows for both health systems and practice groups. As such, Step S120 can include generating an analysis from the set of outputs, wherein the analysis comprises a set of surveillance features characterizing operation of the AI architecture in relation to performance metrics of the healthcare system.

In variations, transformation operations used to transform the set of outputs of Step S110 into a set of surveillance features for the analysis can include: data transformation operations that convert unstructured and semi-structured data (e.g., from reports, from dictation files, from image data, from text data, from other data) into orchestration performance data, in order to analyze workflows and to optimize performance. In examples, operations can include natural language processing operations and/or other processing operations to extract temporal markers, procedure identifiers, modality references, and clinical action terms from narrative reports, followed by normalization and mapping to standardized vocabularies or internal workflow taxonomies. Extracted elements can be time-aligned, aggregated, and correlated with operational metadata—such as order creation, image acquisition, interpretation, verification, and report finalization events—to derive performance metrics reflecting throughput, latency, resource utilization, and exception handling. Additional transformation steps can involve feature engineering, data enrichment, anonymization, and schema conversion to support downstream analytics, dashboards, or predictive models of healthcare system performance in relation to the AI architecture being monitored and refined.

In examples, the set of surveillance features generated for the analysis of Step S120 can be associated with one or more of: continuous monitoring workflows; orchestration of image data; orchestration of test data; orchestration of analytics; orchestration of quality metrics data; orchestration of associated workflows for both health systems and physician practice groups; orchestration of associated workflows for entities in healthcare-associated operations, including payers, life sciences companies, pharmaceutical companies, therapeutics companies, etc.; and other workflows.

In the context of image data orchestration, image data orchestration performance features/metrics generated according to Step S120 can capture how efficiently massive, heterogeneous imaging datasets move from acquisition to interpretation or other downstream processing. In examples, the set of surveillance features can include or be derived from one or more of: ingestion latency, which measures the time required to receive and register incoming studies (e.g., DICOM studies) from scanners or PACS image systems; prefetch accuracy, reflecting how reliably relevant prior exams and series are anticipated and staged before a report is opened; streaming throughput, which quantifies how quickly multi-slice or volumetric images are delivered to the viewer at diagnostic resolution; orchestration success rate, which tracks the proportion of studies correctly routed, normalized, and associated with the right patient, exam, and reporting context; resource utilization efficiency, which assesses CPU, GPU, memory, and network usage during sessions; fault recovery time, which characterizes how quickly a system reroutes or retries after network and/or storage issues; and/or other image data orchestration surveillance features.

In the context of test data (e.g., lab test data) orchestration, test data orchestration performance features/metrics generated according to Step S120 can characterize the efficiency, accuracy, and reliability with which test results are integrated into a workflow to support diagnostic context and clinical correlation. In examples, the set of surveillance features can include or be derived from one or more of: test data ingestion latency, measuring the elapsed time between availability of results in laboratory information systems and their accessibility within the reporting environment; data synchronization accuracy, which quantifies the correctness of subject, encounter, and temporal alignment between laboratory values and imaging studies; contextual availability rate, reflecting the proportion of reports generated with all requisite test parameters present at the time of interpretation; orchestration throughput, which assesses the volume of laboratory result updates processed per unit time; interoperability reliability, which captures the success rate of standards-based data exchanges (e.g., HL7 or FHIR) across clinical systems; exception resolution time, which measures how rapidly missing, delayed, or conflicting test inputs are detected and reconciled; and/or other test data orchestration features.

In the context of analytics and/or other analytics data orchestration, orchestration performance features/metrics generated according to Step S120 can characterize how effectively clinical, imaging, and contextual data are synchronized. In examples, the set of surveillance features can include or be derived from one or more of: data aggregation latency, measuring the time required to assemble imaging metadata, structured findings, priors, and patient context; context completeness, which quantifies the percentage of reports or other returned outputs generated with all required inputs such as modality details, measurements, templates, and historical comparisons present; pipeline throughput, reflecting how many tasks can be processed, enriched, and staged per unit time; orchestration reliability, which tracks the rate of successful data joins and handoffs across PACS, RIS, EHR, and NLP or AI services; scalability efficiency, which evaluates how performance degrades; exception handling time, which measures how quickly missing, conflicting, or corrupted inputs are detected and resolved; and/or other analytics orchestration features.

In the context of workflow monitoring (e.g., continuous workflow monitoring, practice group monitoring, etc.), workflow performance features/metrics generated according to Step S120 can provide a quantitative framework for assessing the efficiency, reliability, and operational integrity of end-to-end clinical, research, development, and/or administrative processes. In examples, the set of surveillance features can include or be derived from one or more of: task cycle time, measuring the elapsed duration between workflow initiation and completion across clinical, diagnostic, and billing stages; handoff latency, quantifying delays introduced at transitions between roles, departments, or information systems; throughput capacity, reflecting the volume of cases, encounters, or work items processed per unit time under normal and peak operating conditions; workflow compliance rate, which characterizes adherence to defined protocols, regulatory requirements, and service-level agreements; exception frequency and resolution time, which capture the incidence and remediation speed of stalled, rerouted, or failed workflow events; resource utilization efficiency, which assesses clinician, staff, and system load relative to output; and/or other workflow monitoring surveillance features in the context of associated workflows for entities in healthcare-associated operations, including payers, life sciences companies, pharmaceutical companies, therapeutics companies, etc.

In the context of quality metrics data orchestration, features generated according to Step S120 can provide a characterization of coordinated collection, normalization, and evaluation of quality-related signals across reporting workflows, data sources, and analytic services to enable continuous performance assessment and governance, for the healthcare system. In examples, the set of surveillance features can include or be derived from one or more of: quality metric aggregation latency, measuring the time required to compute and surface quality indicators following report finalization; cross-system consistency, assessing alignment of quality measures derived from disparate PACS, RIS, NLP, and analytics platforms; metric coverage completeness, quantifying the proportion of reports for which all required quality indicators are successfully derived; and/or other quality-related features associated with regulatory compliance, billing compliance, and/or other compliance.

In the context of healthcare entity performance, surveillance features generated according to Step S120 can provide a characterization of healthcare entity performance in relation to roles and responsibilities. In an example, a healthcare entity performance feature can include or be derived from one or more of: a radiology report completion rate, a dictation reduction percentage, and a speed saved per clinical shift, where examples of performance improvements and various metrics are described above. Generated analysis of current performance can then be used to execute actions (e.g., corrective actions) for maintaining or improving performance for individual contributors and/or groups of contributors, according to Step S130.

In the context of billing accuracy and/or other related performance, surveillance features generated according to Step S120 can include or be derived from one or more of: code validation features (e.g., with features that verify diagnosis and procedure codes against current ICD, CPT, and HCPCS standards); clinical-to-billing consistency check features (e.g., that ensure alignment between documented services, medical necessity, and billed charges); error detection features (e.g., which identify mismatches, missing modifiers, bundling conflicts, and payer-specific policy violations prior to claim submission); eligibility and benefits verification features; charge capture completeness features, audit trail features; and/or other features associated with characterizing billing accuracy performance, where billing performed by the healthcare system is at least somewhat supported using AI architecture.

The features described can thus be used in Step S120 to derive radiology data orchestration and/or other performance features.

Analyses generated in Step S120 can pertain to caretaking-associated entities, at various levels of abstraction in relation to caretaking-associated operations. In variations, analyses can pertain to one or more of: an individual radiologist; an individual clinician; an individual personnel entity; an group of radiologists; a group of clinicians; a caretaking team; a department of a facility; a facility; another caretaking unit; a caretaking specialty; a resource/device (e.g., imaging device, surgical device, robotic apparatus, other medical device, other procedure-performing device, etc.); a category of resource/device; a group of resources/devices; or other entity or object.

Analyses generated in Step S120 can additionally or alternatively pertain to care-receiving entities, at various levels of abstraction. In variations, analyses can pertain to one or more of: a patient; a care-receiving individual who is not a patient; a population of individuals; a demographic; a group of individuals subject to a particular treatment; a group of patients having a clinical indication; a group of individuals who do not have a clinical indication; another grouping of individuals; characterizations of a set of patients of the healthcare system; characterizations of a set of individuals receiving any form of care; and/or any other subset or category of care-receiving entity.

In examples, human patient demographics for which the analyses generated in Step S120 are designed to evaluate can include: human patient demographics (e.g., represented as structured attributes that characterize populations and enable stratified analysis, risk adjustment, and equitable care delivery), where some demographic dimensions include: age distribution (e.g., capturing pediatric, adult, and geriatric cohorts with clinically relevant subranges); biological sex and gender identity(e.g., to support both physiological and sociocultural analyses); race and ethnicity; geographic attributes (e.g., residence, region, or urban-rural classification); socioeconomic indicators (e.g., insurance coverage, income proxies, employment status, and educational attainment); language preference and cultural background; indication type; medical history; medication usage; lifestyle attributes; substance use attributes; and/or other features.

Step S120 can return local analyses (e.g., analyses associated with a location or region). Step S120 can return analyses associated with a larger region (e.g., as a global analysis). Step S120 can return analyses covering longitudinal trends associated with any category. Step S120 can return analyses covering trends based upon societal events (e.g., natural disasters, world events, etc.) and other factors. Step S120 can return analyses associated with changes in government administrations (e.g., at the Federal level, at the state level, at the municipality level, at the district level, at another local level, etc.).

In one example, step S120 can generate an analysis of healthcare system performance in response to administration changes (e.g., federal government administration changes, state government administration changes, local government administration changes), based upon shifts in regulatory priorities, reimbursement policies, and program funding affect operational efficiency, access to care, and clinical outcomes across providers and payers. A representative analysis typically evaluates pre-and post-transition indicators such as coverage rates, service utilization, quality measure performance, cost trends, and administrative burden, while controlling for secular trends and regional variation. In the example, the analysis can evaluate policy-sensitive domains, including, for example, value-based payment participation, Medicaid and marketplace enrollment dynamics, reporting and compliance requirements, and investment in health information technology and interoperability. By correlating policy implementation timelines with longitudinal performance metrics and stratifying results by care setting and patient demographics, such analyses of Step S120 can provide evidence of healthcare system responsiveness, resilience, and unintended consequences.

Step S120 can thus include transforming any baseline analysis into an economic impact estimate or other cost estimate. For instance, returned analyses can project trends in types and/or numbers of procedures being performed, and economic impact estimates can be generated based upon cost tables for various treatments (e.g., provided by Centers for Medicare and Medicaid services (CMS), provided by other cost schedules, etc.). In another example, analyses associated with practitioner/facility/other caretaking entity performance can be transformed into economic impact estimates in relation to percentages of errors made, errors prevented, or other aspects in relation to decisions made during the course of care-providing (e.g., clinically-relevant decisions).

Step S120 can include transforming any baseline analysis into societal health impact analysis. For instance, health trends extracted from baseline analyses of Step S120 can be used to project predominant health issues for a given grouping of individuals. Step S120 can then apply any projections to generating recommendations for guiding public health policy or other policies for improving health of individuals, as described further in relation to Step S130 below.

Analyses of Step S120 can be generated using trained models, where such models are trained iteratively with training data generated from outputs of AI-supported healthcare systems. Models can include multimodal architecture co-trained using various types of data. Exemplary multimodal models can include LLM architecture, SLM architecture, vision encoder architecture, and/or other suitable architecture, embodiments, variations, and examples of which are described in Applications incorporated by reference.

In an example, generation of analyses of Step S120 can be performed by using models that process policy-aware data engineering with longitudinal, multi-level analytics. An exemplary workflow can include: data aggregation, in which structured administrative, clinical, financial, and quality datasets (e.g., claims, EHR extracts, public health indicators, and compliance reports) are temporally aligned (e.g., spanning pre-and post-policy transition periods associated with administrative changes). Policy event encoding is then applied, representing regulatory changes, reimbursement rule updates, or program launches as structured variables or temporal markers that models can reason over. Machine learning models (e.g., causal inference frameworks, time-series models, and difference-in-differences estimators enhanced with representation learning, etc.) can be used to isolate policy effects from background trends, seasonality, and regional variation. The model(s) used can include clustering and stratification architecture for segmenting providers, practice groups, and patient populations to identify heterogeneous responses, in addition to or alternatively to including predictive model architecture for simulating counterfactual scenarios (e.g., expected performance absent a policy change). LLM architecture of the model(s) used to generate analyses in Step S120 can further orchestrate this pipeline by mapping unstructured policy documents to structured policy features, summarizing model outputs into interpretable narratives, and generating traceable explanations aligned with regulatory and operational stakeholders.

Other suitable architecture can be used to return analyses of Step S120, in relation to embodiments, variations, and examples of described in Applications incorporated by reference above.

### 3.2 Method—Monitoring and Surveillance Actions

Step S130 recites: executing an action in response to a feature of the analysis S130, thereby providing surveillance and monitoring of the AI-supported healthcare system. Step S130 functions to guide and/or perform actions in the real world that improve provision of healthcare, improve performance of such AI-supported healthcare systems with respect to safety and reliability (and other features), reduce economic burdens associated with providing good healthcare to individuals, and to provide other benefits. Executing the action in response to a feature of the set of features of the analysis of Step S130 preferably includes adjusting a device configuration of the healthcare system, thereby providing surveillance of the healthcare system; however, as described below, Step S120 can include performing other suitable actions that modulate system performance and/or guide actions in other manners, thereby contributing to better healthcare provision by the associated healthcare system(s).

Adjusting a device configuration in Step S130 can include modifying instructions (e.g., code, etc.) that govern how a subsystem of the healthcare system operates. Such devices can be input devices, output devices, imaging devices, diagnostic devices, test devices, devices used in a workflow of the health care system, and/or other suitable devices. Adjusting a device configuration can be performed proactively, and/or can be performed in response to triggering events. For instance, an output of Step S120 can be used to determine a state of the device that should be monitored for performance issues, and, once the state is detected during subsequent use of the device, modifying how the device operates can be performed in response to the detected state.

In one example related to orchestration latency, latency-associated events detected using the returned surveillance features of Step S120, can be used to anticipate latency issues, and modifying operation of devices can include one or more of: minimizing synchronous dependencies across the acquisition, routing, and rendering pipeline while optimizing data locality and transport efficiency; using low-latency architectures that prioritize event-driven ingestion of image digital objects from modalities; decoupling real-time diagnostic viewing workflows from non-critical downstream processes such as archival persistence, analytics, and quality monitoring; co-locating service functions (e.g., image brokering, orchestration services, etc.) within infrastructure (e.g., PACS and viewer infrastructure) to reduce network round-trip times; streaming larger data payloads (e.g., large image file payloads) using chunked transfer, compression, and progressive rendering techniques to enable time-to-first-image rather than full-study blocking; caching frequently accessed metadata (e.g., study descriptors, series hierarchies, and routing rules, etc.) in a manner that avoids repeated queries against modality worklists; parallelizing prefetching of priors; and other configurational modifications that address latency issues.

Step S130 can thus process outputs of Step S120 in order to perform actions for improving performance of AI-supported healthcare systems; improving orchestration of image data, test data, analytics data, and/or other quality metrics data; improving associated workflows for both health systems and practice groups, as well as other entities (e.g., payers, life sciences companies, pharmaceutical industry entities, etc.); providing full automation production monitoring of AI models and solutions; providing partial automation production monitoring of AI models and solutions partly automated; and providing manual-driven driven production monitoring of AI models and solutions. Step S130 can thus execute actions in the real-world to improve or otherwise maintain proper performance of such AI-supported healthcare systems.

Step S130 can include modifying a structural arrangement of the AI architecture, thereby improving performance of the healthcare system. Variations of Step S130 can thus include adjusting a foundation model or other model component of the AI architecture of the healthcare system.

In variations of model architecture modification, Step S130 can include modifying model, runtime, and/or deployment layers while preserving relevant performance targets. At the model level, modifications can include architecture simplification (e.g., replacing heavy backbones with efficient CNN/ViT variants); operator-level pruning to remove low-salience channels or attention heads; and quantization (e.g., using FP16, INT8, or mixed-precision) to reduce memory bandwidth and accelerator energy per inference; knowledge distillation to transfer accuracy from a larger “teacher” model to a smaller “student” model; and/or other model simplification modifications. Additionally or alternatively, modifications can include implementing cascaded inference to route information through a lighter-weight triage model, while structuring architecture to invoke higher-capacity model architecture for ambiguous or complex cases. Additionally or alternatively, region-of-interest triggering can limit computation performed by the models to relevant regions of interest in data, in order to avoid full-volume processing of all input information.

At the systems level, modifications executed in Step S130 can include performing asynchronous batching and micro-batching, model compilation (e.g., based upon kernel fusion, based upon graph optimization, based upon deployment via TensorRT/ONNX Runtime, etc.), and hardware-aware scheduling (e.g., pinning of operations to graphics processing unit and/or neural processing unit capabilities, dynamic voltage/frequency scaling awareness, NUMA-conscious placement, etc.) in order to reduce tail latency and avoid wasteful data movement. Additionally or alternatively, modifications including edge-cloud partitioning and on-demand model activation can be used to reduce both end-to-end response time and energy draw.

In some embodiments, modifying a structural arrangement or other aspect of model architecture of the healthcare system can thus be performed in conjunction with reducing power usage attributed to the AI architecture of the healthcare system, such that the AI-supported healthcare system can operate in a sustainable manner, from an energy use perspective.

In some embodiments, modifying a structural arrangement or other aspect of model architecture of the healthcare system can additionally or alternatively be performed in conjunction with reducing a latency time attributed to retrieval and transmission of data for the healthcare system, such that the AI-supported healthcare system can operate in a more-efficient manner and reduce workflow bottleneck issues.

In relation to Step S130, the recommended action can pertain to refinement and improvement of AI model architecture implemented by the healthcare system. For instance, the overarching monitoring and surveillance platform described can detect errors in outputs that are consistently corrected for by a human (e.g., radiologist, clinician, therapist) or other entity in the loop of providing care. Step S130 can include notifying entities responsible for managing the AI architecture implemented by the healthcare system, for refinement of such models.

Step S130 can additionally or alternatively include returning one or more recommended actions using a suitable ranking algorithm or other model architecture. The recommended action can pertain to a corrective action for one or more entities involved in caretaking, where the entities are associated with use of the AI-supported healthcare system. As such, executing the action can include applying a corrective action to at least one of an underperforming facility, department, care unit, group of clinicians, and a caretaker associated with the healthcare system. The corrective action can apply to a facility, a department, a care unit, a group of clinicians, an individual caretaker, a category of resources (e.g., medical device, imaging device, etc.), an individual resource, or other suitable entity or device. For instance, determination that a particular entity or group of entities produces suboptimal treatment outcomes, based upon the analyses of Step S120, can be used to inform and execute associated corrective actions in relation to surveillance and monitoring activities. As such, Step S130 can reduce economic impact associated with caretaking-associated errors involving AI-supported healthcare systems.

The recommended action can pertain to an incentivizing action for one or more entities involved in caretaking, where the entities are associated with use of the AI-supported healthcare system. The incentivizing action can reward consistent positive treatment outcomes and can apply to a facility, a department, a care unit, a group of clinicians, an individual caretaker, a category of resources (e.g., medical device, imaging device, etc.), an individual resource, or other suitable entity or device. For instance, determination that a particular entity or group of entities produces consistent positive treatment outcomes, based upon the analyses of Step S120, can be used to inform and execute incentivizing actions in relation to surveillance and monitoring activities. As such, Step S130 can improve healthcare provision associated with caretaking-associated positive outcomes involving AI-supported healthcare systems.

Relatedly, the recommended action or modification to model architecture can, in variations, improve the healthcare system by increasing a report generation accuracy of the healthcare system (e.g., radiology system). Accuracy improvements can be based upon implementing model architecture that tracks report quality as the report is being reviewed or generated (e.g., in relation to automated analysis of findings, measurements, prior sessions with a patient, etc.).

The recommended action can pertain to policy-making guidance. For instance, in relation to detected health trends associated with individuals, demographics, or other groupings of those receiving care, Step S130 can include returning recommended public health policies for improving health of a population and reducing economic impact of poor public health policies.

In examples, executed actions performed according to Step S130 can thus be used to inform and adapt public health policies by translating outputs of Step S120 into actionable population-level insights that balance health outcomes with economic efficiency. For example, outputs of Step S120 can identify geographic or demographic cohorts at elevated risk for preventable conditions, and Step S130 can include execution of responses that enable targeted screening, preventative treatment, or early-intervention programs that reduce downstream hospitalization and productivity loss. In relation to Steps S120 and S130, time-series and causal inference models applied to longitudinal utilization and outcome data can evaluate the real-world impact of policy levers (e.g., reimbursement incentives, access expansions, preventive care mandates), thereby allowing policymakers to refine or retire interventions that underperform relative to cost. Step S130 can additionally include implementing AI-driven surveillance systems, as described, to detect emerging disease patterns or care access gaps earlier than traditional reporting, supporting timely policy adjustments that mitigate large-scale health and economic disruption. By continuously integrating outcomes, cost, and equity metrics into analytic frameworks iteratively, the systems and methods described enable evidence-based public health policies that improve population health while reducing the financial drag associated with late-stage disease and inefficient resource allocation.

In relation to Step S130, the recommended action can thus pertain to guidance and implementation of cost adjustments for a particular treatment or pharmaceutical. For instance, in relation to detected health trends associated with individuals, demographics, or other groupings of entities receiving care, Step S130 can include returning recommended pricing adjustments for a particular treatment or pharmaceutical based upon a set of factors. As such, performance of Step S130 can include applying a cost adjustment for a treatment provided by the healthcare system.

In relation to Step S130, the recommended action can include decommissioning a subsystem of the healthcare system, if surveillance features and/or analysis of Step S120 indicate that the subsystem is operating improperly and should be decommissioned in order to improve performance of the healthcare system.

Step S130 can, however, include generation of and execution of other suitable recommended actions for performing monitoring of AI-supported healthcare systems.

### 4. System

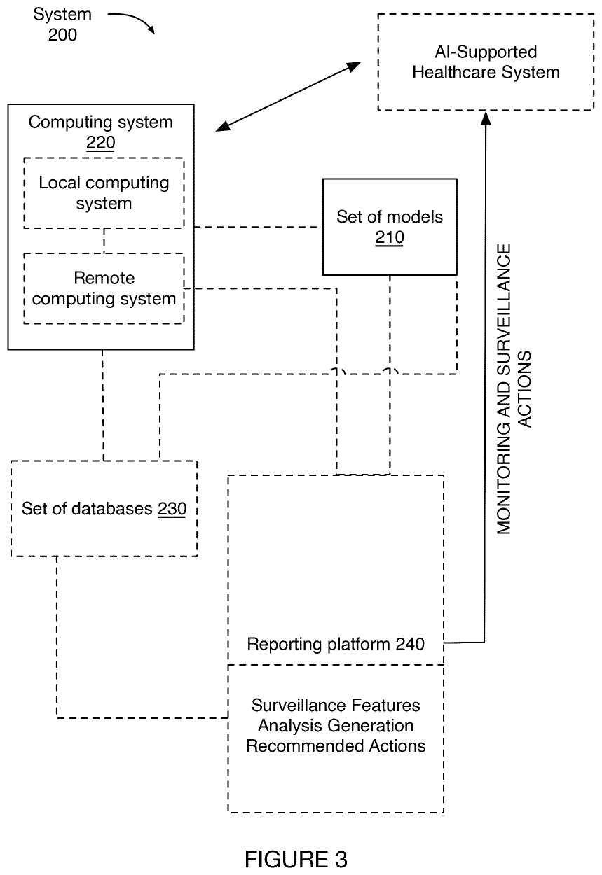

As shown in FIG. 3, a system 200 for surveillance of healthcare systems implementing AI architecture can include a set of models 210 comprising architecture structured to perform one or more steps of the method 100 described in Section 3 above. As shown in FIG. 3, the system 200 can include and/or interface with any or all of: the set of models 210, a computing system 220, a set of databases 230, a user interface (e.g., referred to equivalently herein as an “input interface”), a reporting platform 240 for returning analyses, user devices, and/or any other suitable system components for executing actions responsive to returned outputs. Additionally or alternatively, the system can include any or all of the components as described in any or all of: U.S. application Ser. No. 16/688,623, filed 19 Nov. 2019; U.S. application Ser. No. 17/020,593, filed 14 Sep. 2020; U.S. application Ser. No. 17/690,751, filed 9 Mar. 2022; U.S. application Ser. No. 18/215,354, filed 28 Jun. 2023; U.S. application Ser. No. 17/649,213, filed 28 Jan. 2022; U.S. application Ser. No. 18/374,535, filed 28 Sep. 2023; and U.S. application Ser. No. 18/374,526, filed 28 Sep. 2023, each of which is incorporated in its entirety by this reference.

In embodiments, system 200 comprises instructions stored in non-transitory media that, when executed, perform steps of methods described, including one or more of: retrieving a set of outputs of a healthcare system including artificial intelligence (AI) architecture for generating the set of outputs; generating an analysis associated with the set of outputs; and executing an action in response to a feature of the analysis, thereby providing surveillance of the healthcare system. The system 200 can, however, be structured to perform other suitable method steps.

The set of models 110, function to perform any or all of the processing, generation, training, re-training, transmission, action execution, and/or other steps in the method 100 (e.g., as described above). The models can include architecture for machine learning approaches, classical or traditional approaches, and/or be otherwise configured. The models can include regression, decision tree, LSA, clustering, association rules, dimensionality reduction, neural networks (e.g., CNN; DNN; CAN; LSTM; RNN such as LSTM, GRU, etc.; FNN; encoders; decoders; deep learning models; transformers; etc.), ensemble methods, optimization methods, classification, rules, heuristics, equations (e.g., weighted equations, etc.), selection (e.g., from a library), regularization methods (e.g., ridge regression), Bayesian methods (e.g., Naiive Bayes, Markov), instance-based methods (e.g., nearest neighbor), kernel methods, support vectors (e.g., SVM, SVC, etc.), statistical methods (e.g., probability), comparison methods (e.g., ranking, similarity, matching, distance metrics, thresholds, etc.), deterministics, genetic programs, and/or any other suitable model. The models can include (e.g., be constructed using): a set of input layers (e.g., encoders), output layers (e.g., decoders such as beam search decoders), and/or hidden layers (e.g., connected in series, such as in a feed forward network; connected with a feedback loop between the output and the input, such as in a recurrent neural network; etc.; wherein the layer weights and/or connections can be learned through training); a set of connected convolution layers (e.g., in a CNN); attention mechanisms (e.g., sequence-to-sequence architecture; a set of attention layers and/or self-attention layers; etc.); and/or have any other suitable architecture.

Models can be trained (e.g., pre-trained, retrained, tuned, fine-tuned, etc.), learned, fit, predetermined, untrained, and/or can be otherwise determined. The models can be trained or learned using: supervised learning, unsupervised learning, self-supervised learning, semi-supervised learning (e.g., positive-unlabeled learning), reinforcement learning, transfer learning, Bayesian optimization, fitting, interpolation and/or approximation, backpropagation, and/or otherwise generated. For example, models can be trained based on historical radiology reports (e.g., annotated radiology reports), manually generated radiology reports, synthesized radiology reports, labeled data, unlabeled data, positive training sets, negative training sets, and/or any other suitable set of data. Models can optionally be trained and/or undergo post-processing using: an additional model (e.g., a first model is used to teach a second model), autonomous agents (e.g., while models interact with each other), and/or any other model interactions.

The computing system can include one or more: CPUs, GPUs, neural processing units (NPUs), custom FPGA/ASICS, processors, microprocessors, servers, cloud computing, storage; memory; and/or any other suitable components. The computing system can be local, remote, distributed, or otherwise arranged relative to any other system or module.

The system can include and/or interface with: a reporting platform; a Picture Archiving and Communication System (PACS) and/or alternative image viewing and image storage platform; a speech recognition platform; a radiology worklist; a Radiology Information System (RIS); an electronic medical record (EMR) database; an electronic health record (EHR) database; a Clinical Information System (CIS) platform; a Health Information System (HIS) platform; a Laboratory Information System (LIS) platform; vendor-neutral archive (VNA) components; ontologies (e.g., radiological or other clinical ontology database); and/or any other database, storage, server, and/or software tools. In a specific example, the system includes a reporting platform (including a speech recognition platform and a user interface), wherein the reporting platform receives inputs and/or user actions from a radiologist, and displays a generated radiology report (e.g., determined using one or more models).

In relation to configurations of the system 200, aspects of the system 200 can include modifications and/or be configured for modification of various configurational aspects, based upon returned outputs (e.g., surveillance features, analyses).

In variations, the system 200 can be configured for receiving and executing modified instructions (e.g., code, etc.) that govern system operation. Such system aspects can include devices including input devices, output devices, imaging devices, diagnostic devices, test devices, devices used in a workflow of the healthcare system, and/or other suitable devices.

In the context of orchestration latency, the system 200 can be structured for modification of device operations in the context of one or more of: minimizing synchronous dependencies across the acquisition, routing, and rendering pipeline while optimizing data locality and transport efficiency; using low-latency architectures that prioritize event-driven ingestion of image digital objects from modalities; decoupling real-time diagnostic viewing workflows from non-critical downstream processes such as archival persistence, analytics, and quality monitoring; co-locating service functions (e.g., image brokering, orchestration services, etc.) within infrastructure (e.g., PACS and viewer infrastructure) to reduce network round-trip times; streaming larger data payloads (e.g., large image file payloads) using chunked transfer, compression, and progressive rendering techniques to enable time-to-first-image rather than full-study blocking; caching frequently accessed metadata (e.g., study descriptors, series hierarchies, and routing rules, etc.) in a manner that avoids repeated queries against modality worklists; parallelizing prefetching of priors; and other configurational modifications that address latency issues.

In the context of improved performance, the system 200 can be structured for modification AI architecture, where variations of modifications can include adjusted foundation models or other model components of the AI architecture of the system. In more detail, system 200 modifications can include one or more of: modifications to model, runtime, and/or deployment layers while preserving relevant performance targets. At the model level, modifications can include architecture simplification (e.g., replacing heavy backbones with efficient CNN/ViT variants); operator-level pruning to remove low-salience channels or attention heads; and quantization (e.g., using FP16, INT8, or mixed-precision) to reduce memory bandwidth and accelerator energy per inference; knowledge distillation to transfer accuracy from a larger “teacher” model to a smaller “student” model; and/or other model simplification modifications. Additionally or alternatively, modifications can include implementing cascaded inference to route information through a lighter-weight triage model, while structuring architecture to invoke higher-capacity model architecture for ambiguous or complex cases. Additionally or alternatively, region-of-interest triggering can limit computation performed by the models to relevant regions of interest in data, in order to avoid full-volume processing of all input information.

At the systems level, modified system 200 aspects can include asynchronous batching and micro-batching architecture, architecture for model compilation (e.g., based upon kernel fusion, based upon graph optimization, based upon deployment via TensorRT/ONNX Runtime, etc.), and hardware-aware scheduling (e.g., pinning of operations to graphics processing unit and/or neural processing unit capabilities, dynamic voltage/frequency scaling awareness, NUMA-conscious placement, etc.) in order to reduce tail latency and avoid wasteful data movement. Additionally or alternatively, modifications including edge-cloud partitioning and on-demand model activation can be used to reduce both end-to-end response time and energy draw. As such, modified structural arrangements or other aspect of model architecture of the system 200 can thus be performed in conjunction with reducing power usage attributed to the AI architecture of the healthcare system, such that the AI-supported healthcare system can operate in a sustainable manner, from an energy use perspective. Additionally or alternatively, modified structural arrangements or other aspect of model architecture of the system 200 can be configured for reducing latency time attributed to retrieval and transmission of data for the healthcare system, such that the AI-supported healthcare system can operate in a more-efficient manner and reduce workflow bottleneck issues.

However, the system can be otherwise configured.

### 5. Conclusions

Embodiments of the system and/or method can include every combination and permutation of the various system components and the various method processes, wherein one or more instances of the method and/or processes described herein can be performed asynchronously (e.g., sequentially), contemporaneously (e.g., concurrently, in parallel, etc.), or in any other suitable order by and/or using one or more instances of the systems, elements, and/or entities described herein. Components and/or processes of the following system and/or method can be used with, in addition to, in lieu of, or otherwise integrated with all or a portion of the systems and/or methods disclosed in the applications mentioned above, each of which are incorporated in their entirety by this reference.

Embodiments can implement the above methods and/or processing modules in non-transitory computer-readable media, storing computer-readable instructions that, when executed by a processing system, cause the processing system to perform the method(s) discussed herein. The instructions can be executed by computer-executable components integrated with the computer-readable medium and/or processing system. The computer-readable medium may include any suitable computer readable media such as RAMs, ROMs, flash memory, EEPROMs, optical devices (CD or DVD), hard drives, floppy drives, non-transitory computer readable media, or any suitable device. The computer-executable component can include a computing system and/or processing system (e.g., including one or more collocated or distributed, remote or local processors) connected to the non-transitory computer-readable medium, such as CPUs, GPUs, TPUS, microprocessors, or ASICs, but the instructions can alternatively or additionally be executed by any suitable dedicated hardware device.

As a person skilled in the art will recognize from the previous detailed description and from the figures and claims, modifications and changes can be made to the preferred embodiments of the invention without departing from the scope of this invention defined in the following claims.

What is claimed is:

1. A method comprising:
at a computing system, retrieving a set of outputs of a healthcare system comprising artificial intelligence (AI) architecture for generating the set of outputs;
at the computing system, generating an analysis from the set of outputs, wherein the analysis comprises a set of surveillance features characterizing operation of the AI architecture in relation to performance metrics of the healthcare system; and
executing an action in response to a feature of the set of features, wherein executing the action comprises adjusting a device configuration of the healthcare system, thereby providing surveillance of the healthcare system.

2. The method of claim 1, wherein the set of surveillance features comprises a radiology data orchestration performance feature.

3. The method of claim 2, wherein the radiology data orchestration performance feature is derived from at least one of: latency and orchestration reliability.

4. The method of claim 1, wherein the set of surveillance features comprises an image data orchestration performance feature.

5. The method of claim 4, wherein the image data orchestration performance feature is derived from at least one of: an ingestion latency, a prefetch accuracy, a streaming throughput, an orchestration success rate, an image retrieval rate, a resource utilization efficiency, and a fault recovery time.

6. The method of claim 1, wherein the set of surveillance features comprises a healthcare entity performance feature.

7. The method of claim 6, wherein the healthcare entity performance feature is derived from at least one of: a radiology report completion rate, a dictation reduction percentage, and a speed saved per clinical shift.

8. The method of claim 1, wherein the set of surveillance features comprises a billing accuracy feature.

9. The method of claim 1, wherein the healthcare system comprises an AI radiology platform comprising architecture for automatically generating an impression section of a radiology report.

10. The method of claim 1, wherein the healthcare system comprises an AI radiology platform comprising architecture for automatically annotating radiology image data of a picture archiving and communication system (PACS).

11. The method of claim 1, wherein the healthcare system comprises an AI radiology platform comprising architecture for providing an automatic follow-up service for a patient in response to an actionable finding for the patient.

12. The method of claim 1, wherein executing the action comprises modifying a structural arrangement of the AI architecture, thereby improving the healthcare system.

13. The method of claim 10, wherein improving the healthcare system comprises reducing a power usage attributed to the AI architecture of the healthcare system.

14. The method of claim 10, wherein improving the healthcare system comprises reducing a latency time attributed to retrieval and transmission of data for the healthcare system.

15. The method of claim 10, wherein improving the healthcare system comprises increasing a report generation accuracy of the healthcare system.

16. The method of claim 1, wherein executing the action comprises adjusting a foundation model of the AI architecture of the healthcare system.

17. The method of claim 1, wherein executing the action comprises applying a cost adjustment for a treatment provided by the healthcare system.

18. The method of claim 1, wherein executing the action comprises decommissioning a subsystem of the healthcare system.

19. The method of claim 1, wherein executing the action comprises applying a corrective action to at least one of an underperforming facility, department, care unit, group of clinicians, and a caretaker associated with the healthcare system.

20. The method of claim 1, wherein the AI architecture comprises a multimodal model comprising a large language model (LLM) with a vision encoder coupled to the LLM by an adapter.
